(19) United States
(12) Patent Application Publication
(10) Pub. No.: US 2025/0256131 A1
(43) Pub. Date: Aug. 14, 2025

(54) ULTRASOUND-FOCUSING TRANSDUCER USED IN HIGH INTENSITY FOCUSED ULTRASOUND DEVICE FOR MULTIFOCAL TREATMENT, AND DEVICE COMPRISING SAME

(71) Applicant: ECO DM LAB CO., LTD., Cheongju-si Chungcheongbuk-do (KR)

(72) Inventors: Man Soon YOON, Cheongju-si Chungcheongbuk-do (KR); Young Min PARK, Cheongju-si Chungcheongbuk-do (KR); Jae Hyouk CHOI, Cheongju-si Chungcheongbuk-do (KR); Sang Myeong LEE, Cheongju-si Chungcheongbuk-do (KR)

(21) Appl. No.: 18/857,162

(22) PCT Filed: Apr. 24, 2023
(86) PCT No.: PCT/KR2023/005516

§ 371 Date: Oct. 15, 2024

(30) Foreign Application Priority Data
Apr. 28, 2022 (KR) 10-2022-0053076
May 25, 2022 (KR) 10-2022-0064284

(51) Int. Cl. A61N 7/02; A61N 7/00

(52) U.S. Cl. CPC A61N 7/02; A61N 2007/0034; A61N 2007/0073; A61N 2007/0078

(57) ABSTRACT

The present inventive concept relates to an ultrasound-focusing transducer used in a high intensity focused ultrasound (HIFU) device, and a device comprising the transducer. Two or more piezoelectric elements, in which the ultrasound focal depth changes due to curvature or height difference, are arranged to have a height difference in a piezoelectric element housing to simultaneously treat skin layers having different treatment depths and thereby improve skin wrinkles.

### TECHNICAL FIELD

The present inventive concept relates to an ultrasound-focusing transducer used in a high intensity focused ultrasound (HIFU) device, and a device including the transducer. By using a transducer in which two or more piezoelectric elements, of which an ultrasound focal depth is changed due to a curvature or a step difference, are disposed in a piezoelectric element housing to have a step difference and a device including the transducer, skin layers with different treatment depths are simultaneously treated, thereby providing improvement to skin wrinkles.

### BACKGROUND ART

Ultrasonic technologies are widely used in the medical, industrial, and environmental treatment fields, and recently, with the increasing interest in beauty, various devices using ultrasonic waves have been introduced to prevent skin aging, improve wrinkles, or maintain skin elasticity.

Ultrasonic waves applied to a wrinkled part contract or coagulate skin tissue by transferring heat to a superficial muscular aponeurotic system (SMAS) which is the root cause of wrinkles, thereby improving wrinkles. Since skin care devices use such a principle, devices, which can improve improvement efficiency by focusing ultrasonic waves only on a specific part to be treated, are commercially available. Recently, ultrasonic devices using high intensity focused ultrasound (HIFU) are being used for medical or aesthetic purposes.

Commonly used HIFU piezoelectric elements utilize an optical principle and are mainly processed into a spherical lens shape and used. Light passing through a spherical lens has different focal lengths according to a wavelength, and as a wavelength becomes longer, a focal length is shortened. Due to such an optical principle, when a voltage is applied to a HIFU piezoelectric element, ultrasonic energy generated at thickness mode vibrations is concentrated on a center of a radius of curvature, thereby focusing ultrasonic vibrations. A HIFU piezoelectric element having such a configuration is seated in a plastic housing so that the HIFU piezoelectric element is isolated from water in a transducer container.

Conventional HIFU device transducers are filled with water as a sound wave transmission medium, and a stepper motor is mounted outside the transducer to precisely move the transducer. However, since the stepper motor generates heat when operating, a cooling fan is required as an auxiliary device, which increases volume and power consumption. In addition, as conventional facial treatment transducers, three types of transducers are manufactured to treat a fascia layer (4.5 mm below the skin), a collagen layer (3 mm below the skin), and a dermis layer (1.5 mm below the skin). As an example, FIG. 1 is an image showing an example of the conventional facial treatment device. However, the three types of transducers have the inconvenience of having to replace and mount the transducer each time according to a treatment depth.

In order to solve the inconvenience of having to replace and replace the conventional transducer every time, a HIFU transducer that can treat a dermal layer and a collagen layer with a single transducer has recently been developed. As an example, FIG. 2 is an image showing an example of the HIFU transducer. However, in the HIFU transducer that can treat a dermis layer and a collagen layer with a single transducer, in order to move a focal length according to a treatment depth, six gears are installed: a connection gear for HIFU transfer, a reduction connection gear, an electronic motor connection gear, a connection gear in a transducer, a connection gear for horizontal rotation, and a cam-type connection gear for linear motion conversion and vertical HIFU movement. As such, many gears occupy an excessive volume in the transducer, and a temperature inside the transducer increases due to HIFU transmission. In order to prevent the increase of the temperature, the volume of the transducer should be increased or HIFU output power should be lowered. In addition, since the transducer is driven by a DC motor mounted outside the transducer, power consumption is increased in comparison to the past, and thus when the transducer is developed as a wireless product, there are problems such as limited usage time, heat generation, noise generation, and limited waterproofing.

Therefore, there is a need for the development of a new HIFU transducer with a new structure that improves existing problems by performing treatments simultaneously or alternately with one transducer to shorten treatment time and minimize an incurrence of transducer replacement.

### RELATED ART DOCUMENTS

### Patent Documents

Korean Patent Publication No. 10-1648837
Korean Patent Publication No. 10-1538896

### DISCLOSURE

### Technical Problem

A first object of the present inventive concept is to provide an ultrasound-focusing transducer used in high intensity focused ultrasound (HIFU) for multifocal treatment that is capable of simultaneously performing treatment at various treatment depths.

A second object of the present inventive concept is to provide a device including the transducer provided when the first object is achieved.

### Technical Solution

Provided is an ultrasound-focusing transducer used in a high intensity focused ultrasound device, which includes two or more piezoelectric elements having a curvature, a piezoelectric element housing to which the piezoelectric elements are disposed and bonded, and a transducer housing in which a transmission window is formed and the piezoelectric elements and the piezoelectric element housing are accommodated. An ultrasound focal depth may be changed due to the curvature or a step difference of the piezoelectric elements.

In order to achieve the second object, the present inventive concept provides a high intensity focused ultrasound device including an ultrasound-focusing transducer, a headpiece coupled to the transducer, and a handpiece coupled to the headpiece. The device may transfer heat to a fascia layer, a collagen layer, or a dermis layer below the skin.

### Advantageous Effects

The present inventive concept provides a high intensity focused ultrasound (HIFU) device that is capable of transferring heat to a fascia layer, a collagen layer, or a dermis layer below the skin and a transducer for the device. The device is a device capable of simultaneously performing treatment on two skin layers with different treatment depths and allows treatment to be performed quickly and efficiently.

### DESCRIPTION OF DRAWINGS

FIG. 1 is an image showing an example of a conventional facial treatment device.

FIG. 2 is an image showing an example of a transducer of a conventional facial treatment device.

FIG. 3 shows images showing a conventional piezoelectric element (see FIG. 3A) and a piezoelectric element (see FIG. 3B) of the present inventive concept.

FIG. 4 is an image showing a structure of a piezoelectric element housing to which high intensity focused ultrasound (HIFU) piezoelectric elements of the present inventive concept are disposed and bonded.

FIG. 5 shows images showing a HIFU piezoelectric body in which HIFU piezoelectric elements of the present inventive concept are disposed, bonded, and fixed to a piezoelectric element housing.

FIG. 6 shows images of an actual manufactured shape (see FIG. 6A) and an image (see FIG. 6B) of a HIFU piezoelectric element housing manufactured to narrow an interval between ultrasonic energies emitted from two HIFU piezoelectric elements.

FIG. 7 is an image showing an actually manufactured shape of a connection between a printed circuit board (PCB) and a wire connected to a silver electrode of a HIFU piezoelectric element of the present inventive concept.

FIG. 8 shows images showing a semi-assembled state (see FIG. 8A) of a transducer including a HIFU piezoelectric element of the present inventive concept and a semi-assembled state (see FIG. 8B) of a conventional transducer.

FIG. 9 is an image showing a state in which a lower plate for filling with water is ultrasonically fused to a transducer housing of an ultrasound-focusing transducer.

FIG. 10 is an image showing a state in which, after being filled with water, an ultrasound transmission film is attached to transmit ultrasonic waves to the skin.

FIG. 11 is a view illustrating a headpiece (1) to which a transducer for a HIFU device of the present inventive concept is coupled and a handpiece (2) of a HIFU device of the present inventive concept.

FIG. 12 is an actual image of a hot spot at which acrylic melts due to HIFU energy at a focus area to measure a focus formation state.

FIG. 13 is a table showing a frequency average, a frequency standard deviation, and an impedance standard deviation when a driving frequency of a HIFU device of the present inventive concept is 7.0 MHz.

FIG. 14 is a table showing a frequency average, a frequency standard deviation, and an impedance standard deviation when a driving frequency of a HIFU device of the present inventive concept is 4.0 MHz.

FIG. 15 is a table showing a driving frequency and impedance of a HIFU device of the present inventive concept.

FIG. 16 is a graph showing actual data obtained by measuring a driving frequency and impedance of a device using a Hp4194A impedance-gain phase analyzer.

FIG. 17 is a graph showing a focus shape formed when a HIFU device of the present inventive concept is driven along an X-axis.

FIG. 18 is a schematic view illustrating a depth at a focus is formed while performing treatment using a HIFU device of the present inventive concept.

FIG. 19 is a graph showing a measured ultrasonic output power of a transducer included in a HIFU device of the present inventive concept.

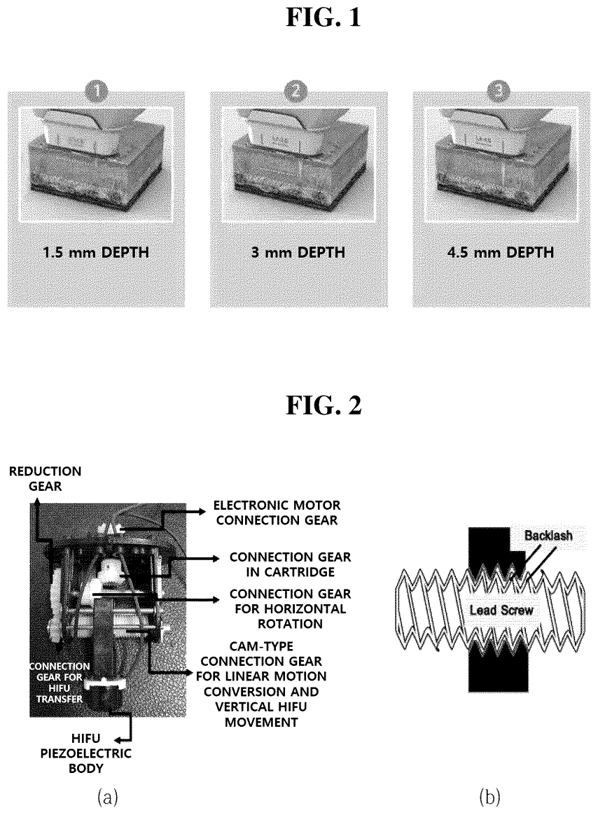

### MODES OF THE INVENTIVE CONCEPT

Hereinafter, embodiments of the present inventive concept will be described in detail with reference to the accompanying drawings so that those skilled in the art may easily carry out the present inventive concept. The embodiments of the present inventive concept are provided to more completely describe the present inventive concept to those skilled in the art. However, the embodiments of the present inventive concept are not intended to limit/restrict the present inventive concept to a specific form, and the described embodiments and configurations shown in the drawings are merely exemplary embodiments of the present inventive concept. In addition, it should be understood that the embodiments of the present inventive concept include all modifications, equivalents, or substitutes included within the spirit and technical scope of the present inventive concept.

Unless defined otherwise, all terms (including technical and scientific terms) used herein have the same meaning as commonly understood by one of ordinary skill in the art to which the present inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, exemplary embodiments of the present inventive concept will be described in detail with reference to the accompanying drawings.

### Embodiments

The present inventive concept provides an ultrasound-focusing transducer used in a high intensity focused ultrasound (HIFU) device that is capable of simultaneously treating skin layers with different treatment depths. The ultrasound-focusing transducer includes two or more piezoelectric elements having an ultrasound focal depth curvature, a piezoelectric element housing to which the piezoelectric elements are disposed and bonded, and a transducer housing in which a transmission window is formed and the piezoelectric elements and the piezoelectric element housing are accommodated. In the ultrasound-focusing transducer, an ultrasound focal depth may be changed due to a curvature or a step difference of the piezoelectric elements.

The ultrasound-focusing transducer may include a piezoelectric linear motor and preferably may include a piezoelectric linear motor for X-axis transfer. The linear motor may cause the piezoelectric elements to move and emit ultrasonic waves.

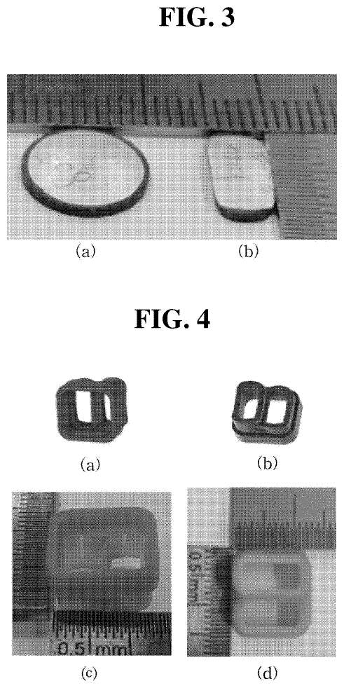

FIG. 3 shows images showing a piezoelectric element (see FIG. 3A) included in a transducer used in a conventional HIFU device and a piezoelectric element (see FIG. 3B) included in a transducer used in the present inventive concept.

Referring to FIG. 3A, a conventional HIFU piezoelectric element has a spherical lens shape, which has a circular outer surface and is convex in an upward direction and concave in a downward direction. In the conventional HIFU piezoelectric element having the spherical lens shape, when ultrasonic waves are generated, ultrasonic energy may be focused on a center of a radius of curvature of an outer diameter, which is an area corresponding to a focal length, and thus focused ultrasonic waves may be focused and radiated on one point, which may cause skin burns during treatment. In addition, when the conventional piezoelectric element is used, side effects such as skin burns may occur due to a mistake or carelessness of an operator, and thus concentration of the operator is required. In order to prevent side effects such as skin burns, an amount of irradiation energy and time of ultrasonic waves may be strictly limited.

Referring to FIG. 3B, in a HIFU piezoelectric element which is a piezoelectric element included in an ultrasound-focusing transducer used in a HIFU device of the present inventive concept, a side surface in a long-axis direction has a smooth straight line shape, and a side surface in a short-axis direction has an arc which is a portion of a circumference of a circle. Specifically, the HIFU piezoelectric element may be a HIFU piezoelectric element having a rectangular shape in which a side surface in a long-axis direction has a smooth straight line shape, and a side surface in a short-axis direction has an arc which is a portion of a circumference of a circle. In the HIFU piezoelectric element of the present inventive concept having the rectangular shape, when ultrasonic waves are generated, although ultrasonic energy is focused on a center of a radius of curvature of an arc of the side surface in the short-axis direction, ultrasonic waves may be widely radiated in the long-axis direction so that ultrasonic energy may not be focused on one point. In addition, ultrasonic energy may not be focused on one point, and ultrasonic waves may be widely radiated in the long-axis direction so that skin damage such as burns may not occur.

The HIFU piezoelectric element, which is the piezoelectric element included in the ultrasound-focusing transducer of the present inventive concept, may be a piezoelectric element of which upper and lower surfaces are coated with silver electrodes, and the silver electrodes may be connected to a conducting wire. In the piezoelectric element, ultrasonic waves may be focused on a center of curvature, and a center position of curvature of each piezoelectric element may be designed differently, thereby making an ultrasound focal depth different.

The HIFU piezoelectric element which is the piezoelectric element included in the ultrasound-focusing transducer of the present inventive concept may be a piezoelectric element having a width of 2.5 mm to 7.0 mm, a height of 8.0 mm to 28.0 mm, and a radius of curvature of 9.0 mm to 15.0 mm and preferably may be a piezoelectric element having a width of 3.5 mm to 6.0 mm, a height of 8.0 mm to 27.0 mm, and a radius of curvature of 10.0 mm to 30.0 mm.

FIG. 4 shows images showing a structure of a piezoelectric element housing to which HIFU piezoelectric elements, which are the piezoelectric elements included in the ultrasound-focusing transducer used in the HIFU device of the present inventive concept, are disposed and bonded. Referring to FIGS. 4A and 4B, in the HIFU piezoelectric elements of the present inventive concept which have a curvature and are disposed and bonded to the piezoelectric element housing of the present inventive concept, a step difference (height difference) may be formed to allow ultrasonic waves to be simultaneously radiated down to different depths below the skin. FIGS. 4C and 4D are images showing an actual manufactured shape.

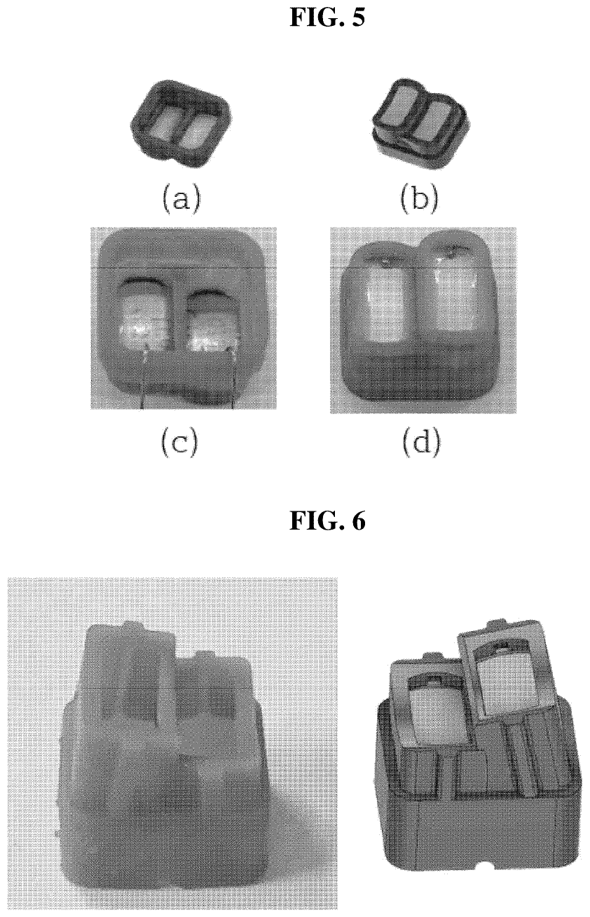

FIG. 5 shows images showing a HIFU piezoelectric body in which HIFU piezoelectric elements, which are piezoelectric elements included in an ultrasound-focusing transducer used in a HIFU device of the present inventive concept, are disposed, bonded, and fixed to a piezoelectric element housing. Referring to FIGS. 5A and 5B, the piezoelectric elements of the present inventive concept having a curvature may have a step difference and may be fixed in parallel to simultaneously radiate ultrasonic waves down to different depths below the skin. FIGS. 5C and 5D are images showing an actual manufactured shape. A wire connected to a silver electrode of the HIFU piezoelectric element may be connected to a printed circuit board (PCB) mounted at an upper portion to waterproof the housing. The PCB may be connected to a circuit unit that is electrically connected to a device including the transducer. The fixed piezoelectric element may be bonded to the housing with epoxy for waterproofing.

FIG. 6 shows images of an actual manufactured shape (see FIG. 6A) and an image (see FIG. 6B) of a piezoelectric element housing included in an ultrasound-focusing transducer of the present inventive concept, wherein the piezoelectric element housing is manufactured to narrow an interval between ultrasonic energies emitted from two piezoelectric elements included in the ultrasound-focusing transducer used in a HIFU device of the present inventive concept. A slope may be made in the arrangement of the two HIFU piezoelectric elements to adjust a focus interval between the piezoelectric elements and form a dual focus. It is preferable to minimize interference of ultrasonic waves of the HIFU piezoelectric element. In addition, the two HIFU piezoelectric elements may be disposed to be inclined to change an ultrasound focal depth. An inclination angle of the HIFU piezoelectric element may be in a range of 0° to 40°, preferably in a range of 0° to 30°. When the inclination angle exceeds 40°, ultrasonic waves emitted from the HIFU piezoelectric elements may overlap each other, which may cause side effects such as burns to the skin. In addition, the piezoelectric element housing may be designed such that the HIFU piezoelectric elements are disposed and bonded to the housing to have a step difference, thereby making ultrasound focal depths different. In addition, ultrasonic waves may be simultaneously applied to skin layers having different depths so that different additional types of piezoelectric elements and separate circuits for generating different frequencies are not required. In addition, since a separate circuit is not required, a burden with respect to designing an ultrasonic device may be reduced. The step difference between the piezoelectric elements may be in a range of 1.0 mm to 4.0 mm, preferably in a range of 1.0 mm to 3.5 mm. When the step difference between the piezoelectric elements is less than 1.0 mm, ultrasonic waves may not be radiated onto different skin layers, and when the step difference between the piezoelectric elements exceeds 4.0 mm, ultrasonic waves emitted from the piezoelectric element at a higher level may not be focused on a position below the skin.

The HIFU piezoelectric element, which is the piezoelectric element included in the ultrasound-focusing transducer used in the HIFU device of the present inventive concept, may have a driving frequency of 2 MHz to 12 MHZ, preferably in a range of 2 MHz to 10 MHz. When the driving frequency is less than 2 MHz, ultrasonic waves may not reach a desired skin layer, and when the driving frequency exceeds 12 MHz, transmitted ultrasonic waves may cause side effects such as burns to the skin.

The driving frequencies of the HIFU piezoelectric elements, which are the piezoelectric elements included in the ultrasound-focusing transducer used in the HIFU device of the present inventive concept, may be the same or different.

In the HIFU piezoelectric elements, which are the piezoelectric elements included in the ultrasound-focusing transducer used in the HIFU device of the present inventive concept, an ultrasound focal depth may be changed due to a curvature or step difference.

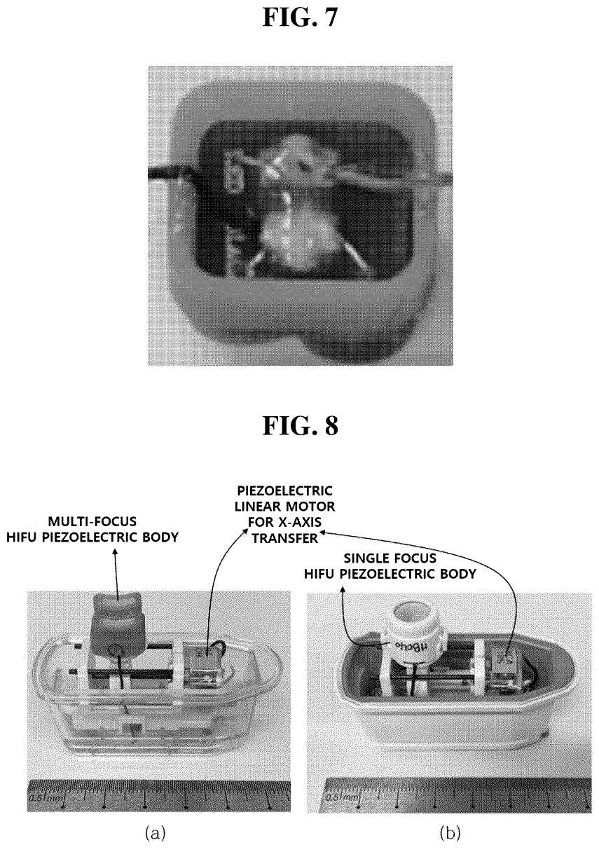

FIG. 7 is an image showing an actual manufactured shape of a connection between a PCB and a wire connected to a silver electrode of a HIFU piezoelectric element which is a piezoelectric element included in an ultrasound-focusing transducer used in a HIFU device of the present inventive concept. Two input terminals and two ground terminals connected inside the piezoelectric element housing may be connected through two wires on the PCB for parallel driving of the HIFU piezoelectric elements. An upper portion of the PCB may be waterproofed by applying epoxy, silicone, or the like after guiding wires connected to a circuit unit of a device including the transducer.

FIG. 8 shows images showing a semi-assembled state (see FIG. 8A) of a transducer including a HIFU piezoelectric body in which HIFU piezoelectric elements, which are piezoelectric elements included in an ultrasound-focusing transducer used in a HIFU device of the present inventive concept, are arranged, bonded, and fixed to a piezoelectric element housing and a semi-assembled state (see FIG. 8B) of a conventional transducer. A transducer for the HIFU device may be designed such that a HIFU focus is formed at a position of 2.5 mm to 4.5 mm below the skin to transfer heat to a fascia layer, a collagen layer, or a dermis layer below the skin.

The transducer for the HIFU device includes a transducer housing forming an exterior of the transducer. The transducer housing may have a structure, in which a transmission window may be formed and the piezoelectric elements and the piezoelectric element housing may be accommodated, and which may be attached to or detached from a handpiece body of a HIFU device. In addition, in the transducer housing, the piezoelectric body of the present inventive concept and the conventional piezoelectric element housing are interchangeable with each other without resulting in any structural change of the transducer housing including a conventional single piezoelectric element having a spherical lens shape.

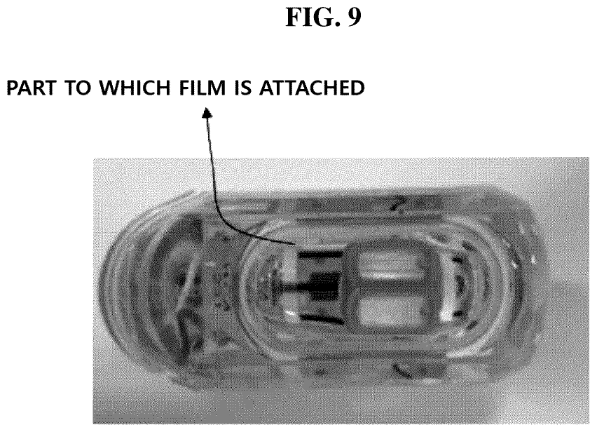

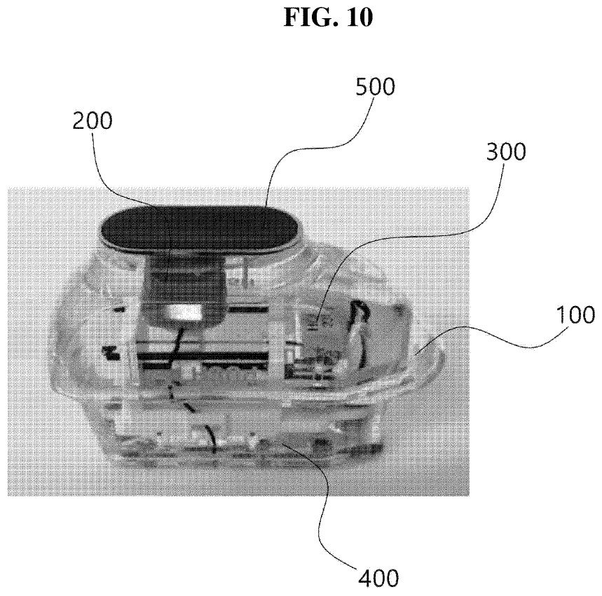

FIG. 9 is an image showing a state in which a lower plate for being filled with water is ultrasonically fused to a transducer housing of an ultrasound-focusing transducer used in a HIFU device of the present inventive concept. FIG. 10 is an image showing a state in which, after the transducer housing of the ultrasound-focusing transducer is filled with water, an ultrasound transmission film is attached to transmit ultrasonic waves to the skin. The transducer housing may include an upper plate or a lower plate, but the present inventive concept is not limited thereto. A sound wave transmission medium, preferably water, may be contained in the transducer housing. In addition, a transmission window may be formed at a lower portion of the transducer housing to efficiently transmit ultrasonic energy generated by HIFU to the skin, and the ultrasound transmission film attached to the transmission window may be included as shown in FIG. 10. In this case, a polymer film commonly used in the art may be used as the ultrasound transmission film. An adhesive portion is formed at an edge of the ultrasound transmission film to be attached to the transmission window. The HIFU piezoelectric body of the present inventive concept having a dual radiation depth may be embedded in the transducer housing, may be controlled by a driving unit of a handpiece coupled to a headpiece including the transducer, and may serve to generate HIFU.

FIG. 10 is an image showing an actual manufactured shape of an ultrasound-focusing transducer, which is used in a HIFU device of the present inventive concept, manufactured according to an exemplary embodiment of the present inventive concept. Referring to FIG. 10, the transducer includes a transducer housing 100, an ultrasonic generator 200 including a HIFU piezoelectric body, a movement control unit 300 which is a piezoelectric linear motor for X-axis transfer, an AC power supply 400, and an ultrasound transmission film 500. The transducer housing 100 accommodates elements that constitute an ultrasonic device and includes an opening, through which ultrasonic waves are transmitted to the skin, at an upper portion. The opening may be shielded with the ultrasound transmission film 500. The ultrasonic generator 200 including the HIFU piezoelectric body is disposed in an internal space of the transducer housing 100. A separation space between the ultrasonic generator 200 and the ultrasound transmission film 500 may be filled with an ultrasonic medium, and the ultrasonic medium preferably may be water. The movement control unit 300 may be attached to a side surface of the ultrasonic generator 200. The movement control unit 300 is a linear motor and is preferably a piezoelectric linear motor for X-axis transfer, which moves the HIFU piezoelectric body along an X-axis. The AC power supply 400 is electrically connected to the ultrasonic generator 200 and provides an AC signal having a resonant frequency to HIFU piezoelectric elements. Since resonant frequencies of two HIFU piezoelectric elements are the same, a single AC signal with one resonant frequency may be applied to drive piezoelectric elements with different effective focal lengths. In addition, the AC power supply 400 may also supply another AC voltage to linearly drive the movement control unit 300.

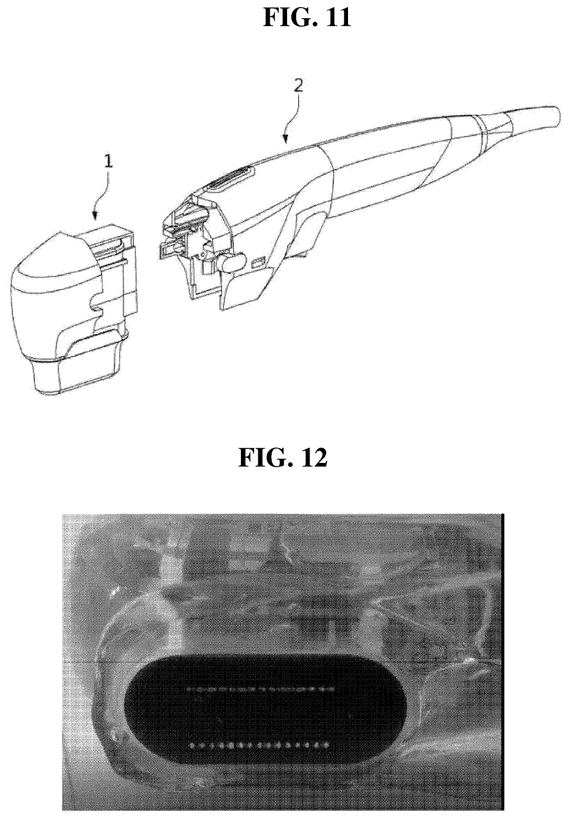

FIG. 11 is a view illustrating a HIFU device of the present inventive concept. The HIFU device may be in a form in which an ultrasound-focusing transducer used in the HIFU device of the present inventive concept is coupled to a headpiece 1 and a form in which a handpiece 2 of the HIFU device is coupled. The handpiece may include a circuit unit, a power supply, and a driving unit. The circuit unit may be electrically connected to a transducer, a HIFU piezoelectric body, and a piezoelectric linear motor for X-axis transfer, and any type of a circuit unit disposed in a typical device may be applied. A signal may be received from the driving unit to apply a voltage to the transducer, the HIFU piezoelectric body, and the piezoelectric linear motor for X-axis transfer. The power supply may be electrically connected to the circuit unit through wires, may serve to supply power to the circuit unit, and may be an external battery, a wireless battery, or an AC power supply according to the embodiments. The driving unit may be electrically connected to the circuit unit and the power supply through wires and may transmit a signal to the circuit unit such that a voltage is applied to the transducer, the HIFU piezoelectric body, and the piezoelectric linear motor for X-axis transfer.

FIG. 12 is an actual image of a hot spot at which acrylic melts due to HIFU energy at a focus area after a device including a transducer of the present inventive concept is driven with an input power of 30 watts to measure a focus formation state. It has been confirmed that dots formed on an ultrasound transmission film matched a thickness of the acrylic due to having a short focal length, and thus it has been confirmed that a thermal coagulation point is formed on a lower surface of the acrylic.

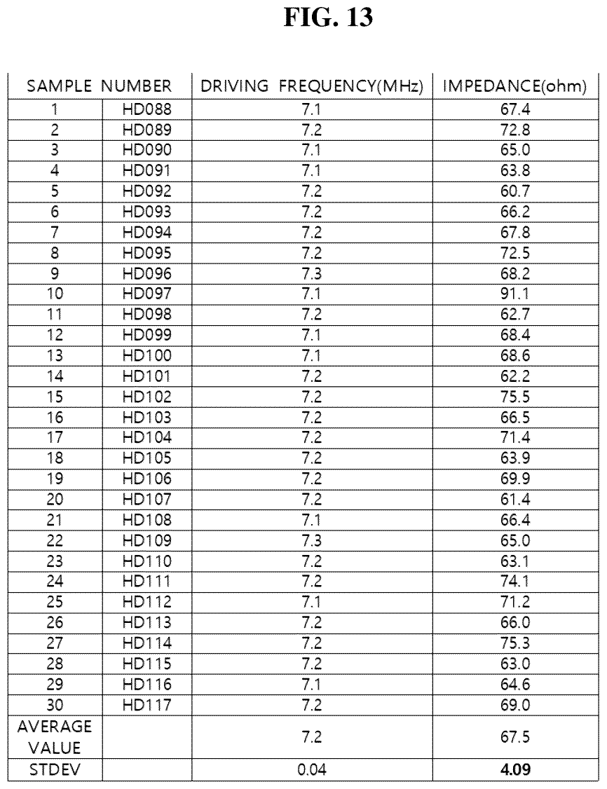

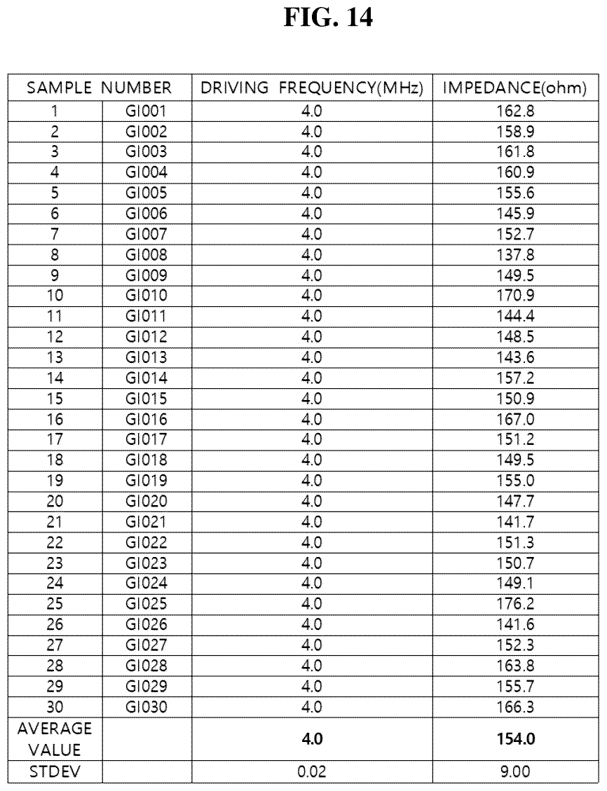

FIGS. 13 and 14 are tables showing a frequency average, a frequency standard deviation, and an impedance standard deviation of a HIFU device of the present inventive concept to confirm the homogeneity of driving frequency and impedance characteristics of a HIFU device including an ultrasound-focusing transducer of the present inventive concept. Referring to FIGS. 13 and 14, it has been confirmed that frequency averages of 7 MHz and 4 MHz transducers of the present inventive concept are 7.2 MHz and 4.0 MHz, respectively, frequency standard deviations thereof are 0.04 and 0.02, respectively, and impedance standard deviations thereof are 4 and 9, respectively. A standard frequency tolerance of a typical medical device company is in a range of +10%. 7 MHz is in a range of 6.3 MHz to 7.7 MHz, and 4 MHz is in a range of 3.6 MHz to 4.4 MHz so that it has been confirmed that a frequency of the HIFU device of the present inventive concept corresponds to a standard frequency. In addition, since HIFU piezoelectric elements having a spherical lens shape, which are generally produced according to a lens processing method, have a frequency standard deviation of 10 or more and an impedance standard deviation of 20 or more, when the HIFU piezoelectric elements are driven in parallel, input power is biased to piezoelectric ceramic at one side, and thus since a driving circuit is separately connected to each piezoelectric ceramic and used, the driving circuit has complexity. In addition, piezoelectric ceramics need to be selected and disposed for parallel driving, which makes mass production difficult. On the other hand, a HIFU piezoelectric element having a rectangular shape of the present inventive concept has low frequency and impedance standard deviation, and thus input power may be uniformly distributed to each HIFU piezoelectric element, which allows mass production without making any separate selection thereof.

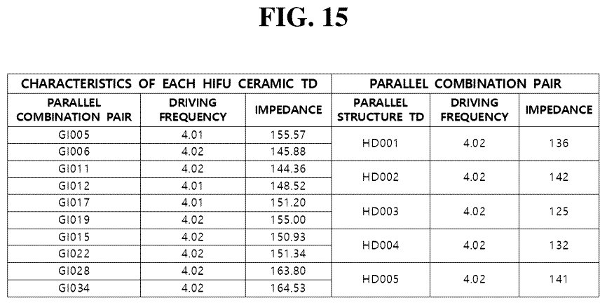

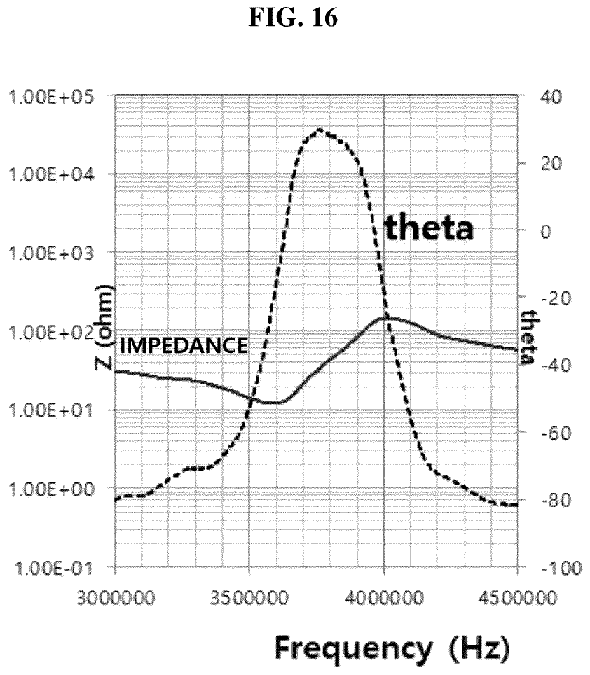

FIG. 15 is a table showing a driving frequency and impedance of a device including a transducer in which a piezoelectric body including parallel-connected HIFU piezoelectric elements is embedded. FIG. 16 is a graph showing actual data obtained by measuring the driving frequency and impedance of the device using a Hp4194A impedance-gain phase analyzer. When a standard deviation is high, there are two driving frequencies. However, in the case of a HIFU device of the present inventive concept, it has been confirmed that driving frequency and impedance values converge to have one driving frequency and impedance value in a similar manner to one piezoelectric element.

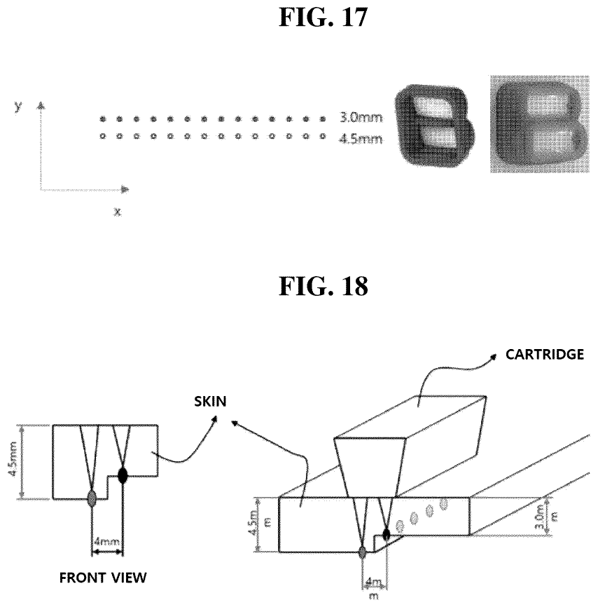

FIG. 17 is a graph showing a focus shape formed when a HIFU device including an ultrasound-focusing transducer manufactured according to an exemplary embodiment of the present inventive concept is driven along an X-axis. Due to the nature of a device applied to a face, as a contact area between the skin and an ultrasound transmission film of the transducer is decreased, the adhesion to the skin may be maximized, thereby allowing side effects such as burns to be avoided. When the contact area is wide, a curved facial portion is difficult to treat, and a space is formed between the skin and a film, which may cause burns. Thus, it is preferable to drive the ultrasound-focusing transducer along the X-axis. When the transducer is driven in parallel (X-axis), two focuses may be formed at a regular interval (3 mm to 5 mm) below the skin. Since the transducer is usually moved over an entire face when the face is treated, a dermal layer and a collagen layer may be stimulated simultaneously when the transducer of the present inventive concept is used.

The HIFU device including the ultrasound-focusing transducer may focus ultrasonic waves down to a depth of 1.0 mm to 6.5 mm below the skin and preferably may focus ultrasonic waves down to a depth of 1.5 mm to 6.0 mm. Thus, the device of the present inventive concept may focus ultrasonic waves to a fascia layer (4.5 mm below the skin), a collagen layer (3 mm below the skin), and a dermis layer (1.5 mm below the skin). In addition, the HIFU device may focus ultrasound waves down to different depths below the skin and specifically may focus ultrasound waves down to a depth of a combination of 1.5 mm and 3.0 mm, 1.5 mm and 4.5 mm, 1.5 mm and 6.0 mm, 3.0 mm and 4.5 mm, 3.0 mm and 6.0 mm, or 4.5 mm and 6.0 mm below the skin.

FIG. 18 is a schematic view illustrating a depth at which a focus is formed during treatment using a HIFU device including an ultrasound-focusing transducer of the present inventive concept. Referring to FIG. 18, a part indicated by a transducer represents an ultrasound transmission film and an ultrasonic generator (HIFU piezoelectric body) of a device including the transducer in which the HIFU piezoelectric body is embedded. The ultrasonic generator moves in an X-axis direction of HIFU piezoelectric elements via a movement control unit. Even though the HIFU piezoelectric elements have the same focal length, the HIFU piezoelectric elements are attached to a piezoelectric element housing to have a step difference, and thus focal lengths to a skin surface may be different. For example, assuming that a first piezoelectric element and a second piezoelectric element among two piezoelectric elements attached to the piezoelectric element housing have a first resonant frequency of 3 MHz, an AC voltage of 3 MHz is applied to the piezoelectric elements. In addition, since the first piezoelectric element and the second piezoelectric element have the same thickness and material, the first piezoelectric element and the second piezoelectric element have the same focal length.

In this case, when the first piezoelectric element is positioned closer to a contact film than the second piezoelectric element, an effective focal length of the first piezoelectric element may be a greater value than that of the second piezoelectric element. For example, due to a step difference, the effective focal length of the first piezoelectric element may be 4.5 mm, and the effective focal length of the second piezoelectric element may be 3 mm.

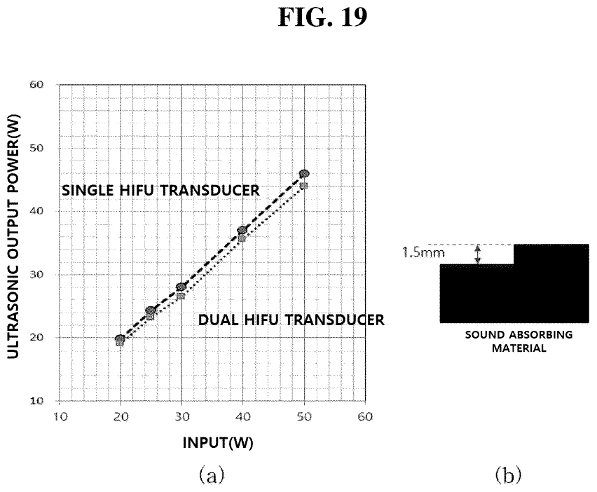

FIG. 19 is a graph showing ultrasonic output power of a transducer included in a HIFU device of the present inventive concept measured using ohmic instrument UPM-DT-1000PA, which is reference equipment utilized by the Ministry of Food and Drug Safety. Data (see FIG. 19A) is obtained by measuring ultrasonic output power when only a first piezoelectric element among two piezoelectric elements included in a HIFU piezoelectric body included in the transducer is driven and a second piezoelectric element is not driven and when the first piezoelectric element and the second piezoelectric element are driven simultaneously. When measured, the ultrasonic output power is measured by changing input Watt. Referring to the graph of FIG. 19A, it has been confirmed that when only the first pressure element is driven according to input power, the ultrasonic output power increases linearly according to the input power. In addition, since output power should be measured at different focuses to simultaneously form two focuses with one input power and measure ultrasonic output power, as results of measuring ultrasonic output power generated from the first pressure element and the second pressure element according to the input power after installing a sound absorbing material having a step difference as shown in FIG. 21B, it has been confirmed that, due to parallel driving, the sum of the output powers of the two HIFU transducers linearly increased according to the input power. Thus, it has been confirmed that power is uniformly distributed to the first pressure element and the second pressure element of the HIFU piezoelectric element.

### DESCRIPTION OF REFERENCE NUMERALS

1: headpiece to which transducer is coupled
2: handpiece
100: transducer housing
200: ultrasonic generator
300: movement control unit
400: AC power supply
500: ultrasound transmission film

1. An ultrasound-focusing transducer used in a high intensity focused ultrasound device, comprising:
two or more piezoelectric elements having a curvature;
a piezoelectric element housing to which the piezoelectric elements are disposed and bonded; and
a transducer housing in which a transmission window is formed and the piezoelectric elements and the piezoelectric element housing are accommodated,
wherein an ultrasound focal depth is changed due to the curvature or a step difference of the piezoelectric elements.

2. The ultrasound-focusing transducer of claim 1, wherein the piezoelectric element has a width of 3.5 mm to 6.0 mm, a height of 8.0 mm to 26.0 mm, and a radius of curvature of 10.0 mm to 30.0 mm.

3. The ultrasound-focusing transducer of claim 1, wherein the piezoelectric element is disposed to be inclined at an angle of 0° to 30°.

4. The ultrasound-focusing transducer of claim 1, wherein the piezoelectric element housing is disposed such that the piezoelectric elements have the step difference.

5. The ultrasound-focusing transducer of claim 4, wherein the piezoelectric elements have a step difference of 1.0 mm to 3.5 mm.

6. The ultrasound-focusing transducer of claim 1, wherein the piezoelectric elements have a driving frequency of 2 MHz to 10 MHz.

7. The ultrasound-focusing transducer of claim 1, wherein driving frequencies of the piezoelectric elements are the same or different.

8. The ultrasound-focusing transducer of claim 1, comprising a piezoelectric linear motor for X-axis transfer.

9. The ultrasound-focusing transducer of claim 1, wherein an ultrasound transmission film is attached to the transmission window of the transducer.

10. A high intensity focused ultrasound device comprising:
an ultrasound-focusing transducer;
a headpiece coupled to the transducer; and
a handpiece coupled to the headpiece.

11. The high intensity focused ultrasound device of claim 10, wherein ultrasonic waves are focused down to a depth of 1.5 mm to 6.0 mm below skin.

12. The high intensity focused ultrasound device of claim 10, wherein the device allows two skin layers having different treatment depths to be simultaneously treated.

13. The high intensity focused ultrasound device of claim 12, wherein a treatment depth of the device is a combination of 1.5 mm and 3.0 mm, 1.5 mm and 4.5 mm, 1.5 mm and 6.0 mm, 3.0 mm and 4.5 mm, 3.0 mm and 6.0 mm, or 4.5 mm and 6.0 mm below skin.
